(19) United States
(12) Patent Application Publication
(10) Pub. No.: US 2010/0316592 A1
(43) Pub. Date: Dec. 16, 2010

(54) AQUEOUS PREPARATION COMPRISING eMIP AS ACTIVE INGREDIENT

(75) Inventors: Teruo AKUTA, Tokyo (JP); Yoshiko Aoki, Tokyo (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ECI, Inc., Tokyo (JP)

(21) Appl. No.: 12/502,959

(22) Filed: Jul. 14, 2009

(30) Foreign Application Priority Data
Jun. 16, 2009 (JP) 2009-143120

(51) Int. Cl. A61K 38/16

(52) U.S. Cl. 424/85.1

(57) ABSTRACT

An objective of the present invention is to provide aqueous preparations comprising eMIP, a derivative of macrophage inflammatory protein 1α (MIP-1α) which has an immunopotentiation activity, and stabilizer(s). The present inventors conducted dedicated studies to achieve the above-described objective. As a result, the present inventors discovered that eMIP degradation is suppressed by addition of at least one or more additives selected from sodium chloride, L-histidine, L-arginine, L-arginine hydrochloride, L-lysine hydrochloride, citric acid, and sodium edetate (EDTA). Furthermore, the present inventors demonstrated that phosphate buffer of pH 5 to pH 7 is a preferable pH adjuster for the aqueous eMIP preparations of the present invention.

### FIELD OF THE INVENTION

The present invention relates to aqueous preparations comprising eMIP, which is a macrophage inflammatory protein 1α (MIP-1α) derivative having an immunopotentiating effect, and stabilizer(s).

### BACKGROUND OF THE INVENTION

In the treatment of cancers, immunological abnormalities, and other diseases, a routinely used method is to isolate genes of bioactive proteins from a living body, introduce these genes into an expression system, culture the cells to obtain the proteins on a large scale, purify the obtained proteins, and use these purified proteins for treatment.

Macrophage inflammatory protein 1α (MIP-1α) consisting of 70 amino acids is a ligand of CCR1 and CCR5, which are C-C chemokine receptors. MIP-1α is known to have the activity of causing migration of various lymphocytes which express the above receptors, including peripheral blood monocytes, dendritic cell precursors, T lymphocytes, and NK cells (Hideki Nakano, Saibou Kougaku (cell technology), Vol. 19, No. 9, 1304-1310 (2000)). Furthermore, it has been reported that T lymphocytes and dendritic cells are recruited to the proximity of cancer cells made to express MIP-1α protein gene and that the protein enhances interferon induction from T lymphocytes, thus suppressing cancer metastasis (Yoneyama H. et al., J. Exp. Med., 193 (1), 35-49 (2001); Zhang Y. et al., J. Natl. Cancer Inst., 96, 201-209 (2004); McKay P F. et al., Eur. J. immunol., 34, 1011-1020 (2004)). In addition, MIP-1αinjection was reported to result in cancer shrinkage or disappearance (WO 2004/037288 A1, herein below, referred to as “Patent Document 1”). Thus, attempts of using MIP-1α for cancer have been assessed.

However, MIP-1α precipitates easily at a concentration of even 10 mg/ml and this poor solubility of MIP-1α, namely its tendency to aggregate and precipitate easily, was considered to be a problem when using for therapy. One approach that has been taken to solve this problem is alteration of the structural gene for MIP-1α so as to substitute alanine for aspartic acid at position 26 and remove N-terminal alanine to give a sequence of 69 amino acids starting with serine. The protein yielded through this alteration is called “BB 10010”, and was assessed in clinical studies aiming at protecting bone marrow during cancer chemotherapy (E. Marshall et al., European Journal of Cancer, 34 (7), 1023-1029 (1998); Hal E. Broxmeyer et al, Blood Cells, Molecules and Diseases, 24 (2), 14-30 (1998)).

The above BB10010 was named “eMIP” and was developed by the present inventors. eMIP was demonstrated to have reduced aggregating activity as well as the activities of inducing chemotaxis, increasing intracellular calcium level, and other activities similar to MIP-1α. Furthermore, intravenous administration of eMIP following local cancer irradiation has demonstrated a remarkable cancer growth suppression effect and abscopal effect, which is the effect of suppressing the growth of cancers located far from the irradiation site (WO 2006/080171 A1, herein below, referred to as “Patent Document 2”).

In addition, BB10010, namely eMIP, is known to have the activity of increasing the level of dendritic cell precursors in blood (Patent Document 1).

Aqueous preparations of BB10010, namely eMIP, are described in, for example, Patent Document 2. This document describes that physiological saline, or an isotonic solution containing glucose or other auxiliary agent, is used for the aqueous preparations, and that they may be combined with an appropriate solubilizing agent, for example, alcohol, polyalcohol, or a non-ionic surfactant (for example, Polysorbate 80™). However, the document does not mention the issue of eMIP stability in aqueous preparations and ways of overcoming it.

### SUMMARY OF THE INVENTION

Conventional protein preparations are commonly prepared utilizing a solubilizing method specific to each protein, and are ultimately used after dissolving in a buffer. eMIP is soluble as a result of introducing amino acid substitutions to the MIP-1α protein which aggregates and precipitates very easily. However, eMIP still potentially retains the MIP-1αprotein's property of aggregating easily, and therefore, detailed assessments are required when formulating eMIP into injections or drip infusions.

eMIP sometimes precipitated when dissolved in a conventional injection buffer, comprising acetic acid, citric acid, phosphoric acid, or a salt thereof. Furthermore, as disclosed herein by the present inventors, a problem encountered was that eMIP degrades over time even when the pH of the solvent is maintained neutral. In addition, eMIP was found to be readily adsorbed onto plastic or glass surface, which makes handling difficult.

The present invention was achieved in view of the above circumstances. An objective of the present invention is to provide aqueous preparations comprising eMIP, which is a derivative of macrophage inflammatory protein 1α (MIP-1α) having an immunopotentiation activity, and stabilizer(s).

The present inventors conducted dedicated studies to achieve the above-described objective.

First, the present inventors assessed the contribution of pH to the solubility of eMIP in aqueous preparations. The result showed that, in the absence of an additive, eMIP formed a precipitate under the weakly acidic condition of pH 5 to 6, while it did not when pH was kept around neutral (pH 7.2 to 7.4). Furthermore, a storage/stability test showed that eMIP easily degrades around pH 7.

Next, additives were added to aqueous eMIP solutions at pH 5 or 7 and the solubility and stability of eMIP were assessed for the purpose of investigating solvent conditions that suppress eMIP degradation. The result demonstrated that eMIP degradation is suppressed by the addition of at least one or more additives selected from sodium chloride, L-histidine, L-arginine, L-arginine hydrochloride, L-lysine hydrochloride, citric acid, and sodium edetate (EDTA). It was also revealed that a phosphate buffer between pH 5 and 7 was suitable as a pH adjuster for the present invention's aqueous eMIP preparations.

Furthermore, additives that suppress the adsorption of eMIP onto plastic or glass surface were assessed by the methods described in EXAMPLE 2. The result showed that the adsorption of eMIP onto plastic or glass surface can be prevented by adding a low concentration of a polysorbate, which is a non-ionic surfactant.

Specifically, the present inventors succeeded in preparing stable aqueous preparations comprising eMIP and completed the present invention.

More specifically, the present invention provides the following [1] to [3]:

[1] An aqueous preparation comprising eMIP as an active ingredient, wherein the preparation comprises one or more additives selected from L-histidine, L-arginine, L-arginine hydrochloride, L-lysine hydrochloride, sodium chloride, citric acid, and sodium edetate (EDTA), and wherein the pH of the preparation is adjusted to 5 to 7.
[2] The aqueous eMIP preparation of [1], wherein the pH adjustment is achieved by using a phosphate buffer.
[3] The aqueous eMIP preparation of [1] or [2], wherein the concentration of eMIP as an active ingredient is within the range of 0.01 to 6.5 mg/ml and the concentration of the additives is within the range of 5 to 15 mg/ml in the aqueous preparation.

### BRIEF DESCRIPTION OF THE DRAWINGS

FIG. 1 is a chromatogram for degradation products after addition of sodium chloride. The chromatogram was obtained immediately after the addition of sodium chloride.

FIG. 2 is a chromatogram for degradation products after addition of sodium chloride. The chromatogram was obtained after one week of preservation at 40° C. in the dark.

### DETAILED DESCRIPTION OF THE INVENTION

The present invention relates to aqueous preparations comprising eMIP, a derivative of macrophage inflammatory protein 1α (MIP-1α) having an immunopotentiating activity, and stabilizer(s).

Herein, “eMIP” refers to a substance that has the activities of inducing chemotaxis, increasing intracellular calcium level, and other activities similar to macrophage inflammatory protein 1α (MIP-1α), and produces, upon intravenous administration after local cancer irradiation, a remarkable cancer growth-suppressing effect and abscopal effect, which is the effect of suppressing the growth of cancers located far from the irradiation site.

“eMIP” is a mutant MIP-1α that consists of 69 amino acids starting with Ser at the amino terminus and having a substitution at position 26 of MIP-1α from Asp to Ala, and is known to have a significantly improved anti-aggregation property as well as an activity comparable to that of the wild type (E. Marshall et al., European Journal of Cancer, 34 (7), 1023-1029 (1998)). In the present invention, “eMIP” can be prepared by methods known to those skilled in the art, more specifically, by the method described in WO 2006/080171; however, the preparation methods are not limited thereto.

Additives can be added to the aqueous preparations of the present invention, in order to stabilize eMIP in the solvent. The additives used in the present invention include, for example, at least one or more additives selected from sodium chloride, D-mannitol, D-sorbitol, purified white sugar (sucrose), glycine, L-alanine, L-histidine, L-arginine, L-arginine hydrochloride, sodium L-glutamate, L-aspartic acid, L-lysine hydrochloride, citric acid, and sodium edetate (EDTA). More preferably, the additives include at least one or more additives selected from L-histidine, L-arginine, L-arginine hydrochloride, L-lysine hydrochloride, citric acid, and sodium edetate (EDTA). Furthermore, non-ionic surfactants may be added to suppress the adsorption of eMIP onto plastic or glass surfaces. Herein, preferred non-ionic surfactants include polysorbates, and more preferred non-ionic surfactants include, for example, Polysorbate 80™.

The pH of the aqueous preparations of the present invention may be adjusted to 5 to 7 with a buffer. Such a buffer includes phosphate buffer (phosphate+sodium phosphate), acetate buffer (acetic acid+sodium acetate), citrate buffer (citric acid+sodium citrate), borate buffer, tartrate buffer, and Tris buffer. More preferably, a phosphate buffer can be used to adjust the pH.

The concentration of additive is not particularly limited; however, when at least one or more additives selected from L-histidine, L-arginine, L-arginine hydrochloride, L-lysine hydrochloride, sodium chloride, citric acid, and sodium edetate (EDTA) are used, the additive concentrations are preferably in the range of 5 to 15 mg/ml (the weight % concentration is within the range of 0.5 to 1.5%). Alternatively, when polysorbates are used as the additives, the weight % concentrations of the additives are preferably within the range of 0.005 to 0.1%.

The concentration of eMIP, which is an active ingredient, is also not particularly limited; however, the content is preferably in the concentration range of 0.01 to 6.5 mg/ml.

The aqueous preparations of the present invention can be formulated according to conventional methods (for example, Remington's Pharmaceutical Science, latest edition, Mark Publishing Company, Easton, U.S.A), and may contain pharmaceutically acceptable carriers and additives in addition to the additives described above. Such pharmaceutically acceptable carriers and additives include surfactants, excipients, coloring agents, flavoring agents, preservatives, stabilizers, buffering agents, suspension agents, isotonizing agents, binding agents, disintegrating agents, lubricants, fluidizing agents, and corrigents. Without being limited to the above examples, other conventional carriers can be appropriately used. More specifically, such carriers include light anhydrous silicic acid, lactose, crystalline cellulose, mannitol, starch, carmellose calcium, carmellose sodium, hydroxypropylcellulose, hydroxypropyl methylcellulose, polyvinylacetal diethylaminoacetate, polyvinylpyrrolidone, gelatin, medium-chain triglyceride, polyoxyethylene hydrogenated castor oil 60, white soft sugar (sucrose), carboxymethyl cellulose, cornstarch, and inorganic salts.

The present invention relates to immunopotentiators comprising the aqueous preparations described above. The immunopotentiators of the present invention are provided as preventive agents for cancer metastasis or therapeutic agents for cancer.

The immunopotentiators of the present invention can be administered to a patient orally or parenterally. The administration is preferably carried out parenterally. Specifically, such administration methods include injection, intranasal administration, intrapulmonary administration, and percutaneous administration. As examples of administration by injection, the immunopotentiators of the present invention can be administered locally or systemically by intravenous, intramuscular, intraperitoneal, or subcutaneous injection. Moreover, the methods of administration can be appropriately selected according to the age and symptoms of the patients. The dose can be selected, for example, from the range of 0.0001 mg to 1,000 mg per kilogram of body weight per administration. Alternatively, doses can be selected, for example, from the range of 0.001 to 100,000 mg/body for each patient. However, the dose of an immunopotentiator of the present invention is not limited to the doses described above.

The present invention demonstrated that eMIP degradation was suppressed by adding an above-described additive(s) to aqueous eMIP preparations. Specifically, the additives used in the present invention greatly contribute to the stability of aqueous eMIP preparations. All patents, published patent applications, and publications cited herein are incorporated by reference in their entirety.

### EXAMPLES

Herein below, the present invention will be specifically described using examples, however, the technical scope of the present invention is not to be construed as being limited thereto.

### Example 1

### Assessment of pH of Aqueous Preparations Comprising eMIP

First, the present inventors assessed the contribution of pH to the solubility of eMIP in aqueous preparations.

When eMIP was dissolved at a concentration of 1.0 mg/ml in the above-described buffers at various pHs, a precipitate was formed under the weakly acid condition of pH 5 to 6. However, when the pH was maintained around neutral and eMIP was preserved for a long period of time, no precipitation was seen. Thus, the pH adjustment range was tentatively fixed to pH 7.2 to 7.4.

A preservation/stability test conducted thereafter showed that eMIP easily degraded around pH 7.

### Example 2

### Assessment of the Contribution of Additives to the Stability of eMIP in Aqueous Preparations

Next, additives were added to aqueous solutions at pH 5 or pH 7, and the solubility and stability of eMIP were assessed for the purpose of investigating solvent conditions that suppress eMIP degradation.

The solubility and stability were assessed by the following procedure.

### (1) Method of Preparing Samples:

(a) The solvent sodium chloride-containing phosphate buffer dissolving “eMIP” was changed to 20 mM phosphate buffer (pH 7) using an ultrafiltration filter (Sartorius VIVASPIN20) to prepare a solution of about 2 mg/ml (pH 7).

(b) 20 mM sodium dihydrogenphosphate (Wako Pure Chemical Industries, Japanese Pharmaceutical Excipients) solution and 20 mM dibasic sodium phosphate hydrate (Wako Pure Chemical Industries, Japanese Pharmacopoeia Grade) solution were combined together, and the pH was adjusted to 5 or 7. To the two types of solutions, each of the various additives (the 36 types of additives shown in Tables 1 and 2) was added to be twice the amount indicated in Table 1 or 2. Each solution was prepared such that the pH became precisely 5 or 7 after additive addition.

When the solution could not be adjusted to pH 5 or pH 7 by this method, a small volume of aqueous solutions of 100 mM phosphoric acid (Nacalai Tesque, special grade) or 100 mM sodium hydroxide (Nacalai Tesque, special grade) was added to adjust the pH of solution to 5 or 7.

TABLE 1
Concentrations of various additives (1)

| | | | Amount added(*) |
|---|---|---|---|
| No. | Category | Additive | (part by weight) |
| 1 | Salt | Sodium chloride | 10 |
| 2 | | Calcium chloride hydrate | 10 |
| 3 | | Magnesium chloride | 10 |
| 4 | Sugar | D-Mannitol | 10 |
| 5 | | D-Sorbitol | 10 |
| 6 | | Fructose | 10 |
| 7 | | Lactose hydrate | 10 |
| 8 | | Sucrose | 10 |
| 9 | | Glucose | 10 |
| 10 | | Glycerin | 10 |
| 11 | Amino acid | Glycine | 10 |
| 12 | | L-Alanine | 10 |
| 13 | | L-Histidine | 10 |
| 14 | | L-Arginine | 10 |
| 15 | | L-Arginine hydrochloride | 10 |
| 16 | | Sodium L-glutamate | 10 |
| 17 | | L-Aspartic acid | 1 |
| 18 | | L-Cysteine | 10 |
| 19 | | L-Lysine hydrochloride | 10 |

(*)Parts by weight of an additive per one part by weight of ECI301

TABLE 2
Concentrations of various additives (2)

| | | | Amount added(*) |
|---|---|---|---|
| No. | Category | Additive | (part by weight) |
| 20 | Surfactant | Sorbitan sesquioleate | 10 |
| 21 | | Polyoxyethylene | 1 |
| | | hydrogenated castor | |
| | | oil 60 | |
| 22 | | Polyoxyethylene sorbitan | 10 |
| | | monolaurate | |
| 23 | | Polysorbate 20 | 10 |
| 24 | | Polysorbate 80 | 10 |
| 25 | | Macrogol 400. | 10 |
| 26 | | Polyoxyethylene (160) | 10 |
| | | polyoxypropylene (30) glycol | |
| 27 | Anti-oxidant | Ascorbic acid | 10 |
| 28 | | Sodium hydrogen sulfite | 10 |
| 29 | | Sodium sulfite | 10 |
| 30 | | α-Thioglycerin | 10 |
| 31 | | Cysteine hydrochloride | 10 |
| 32 | | Dried sodium sulfite | 10 |
| 33 | | Citric acid hydrate | 10 |
| 34 | | Sodium thioglycolate | 10 |
| 35 | | Sodium pyrosulfite | 10 |
| 36 | Chelating agent | Sodium edetate (EDTA) | 10 |

(*)Parts by weight of an additive per one part by weight of ECI301

(c) An equal volume of the solution prepared as described in (a) was added to each of the solutions prepared as described in (b) and mixed. The resulting mixtures were aliquoted (1 ml) to vials (DAIWA SPECIAL GLASS Co., Ltd., borosilicate glass vial). Caps were placed on the vials and screwed fully tight. Thus, the samples were prepared.

### (2) Storage Conditions:

The samples prepared by the preparation method described above were preserved for one week at 40° C. in the dark.

### (3) Test for Degradation Product:

Two types of mobile phases (mobile phases A and B) were prepared to carry out high performance liquid chromatography (hereinafter referred to as “HPLC”).

Mobile phase A was prepared by adding 0.5 ml of trifluoroacetic acid (Wako Pure Chemical Industries, high performance liquid chromatography grade) to 1,000 ml of purified water.

Mobile phase B was prepared by adding 200 ml of purified water and 0.5 ml of trifluoroacetic acid to 800 ml of acetonitrile (Nacalai Tesque, high performance liquid chromatography grade).

900 μl of 0.01% Polysorbate 80™ solution was added to 100 μl each of 36 types of samples preserved under the above-described preservation conditions to prevent the adsorption onto plastic or glass surface, thus giving HPLC sample solutions.

The 0.01% Polysorbate 80™ solution was prepared by weighing 1 g of Polysorbate 80 and adding purified water to it to make the volume 100 ml, and then adding purified water to a 1-ml aliquot of this solution to make the volume 100 ml.

As reverse phase HPLC column, Symmetry300™ C-18 (Waters; I.D. 4.6 mm×L. 150 mm, 5 μm) was used. A sample solution was loaded (0 min) and then the above-described mobile phases A and B were eluted at 1 ml/min according to the programmed time scheme shown in Table 3. Peaks between the retention time of 5 minutes and 45 minutes were measured by automatic integration method. Upon completion of one cycle of the programmed time scheme, another arbitrary sample solution was loaded and the same programmed time scheme was commenced. By this method, the rate of generation of degradation product generated from eMIP was assessed at the time of addition of the 36 types of additives, and after adding the additive and preserving for one week at 40° C. in the dark.

TABLE 3
Programmed time scheme for mixing mobile phases A and B

| Time (min) | Mobile phase A (%) | Mobile phase B (%) |
|---|---|---|
| 0 | 80 | 20 |
| 40 | 20 | 80 |
| 45 | 20 | 80 |
| 46 | 80 | 20 |

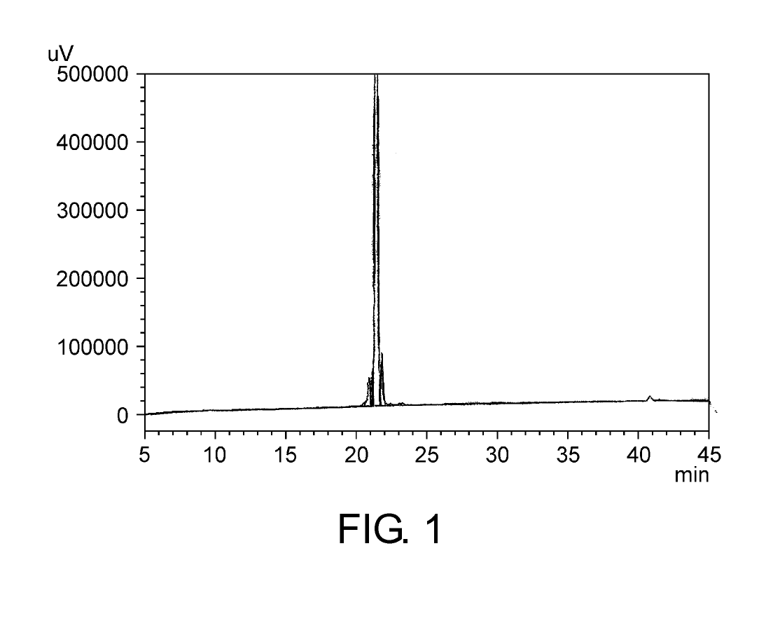

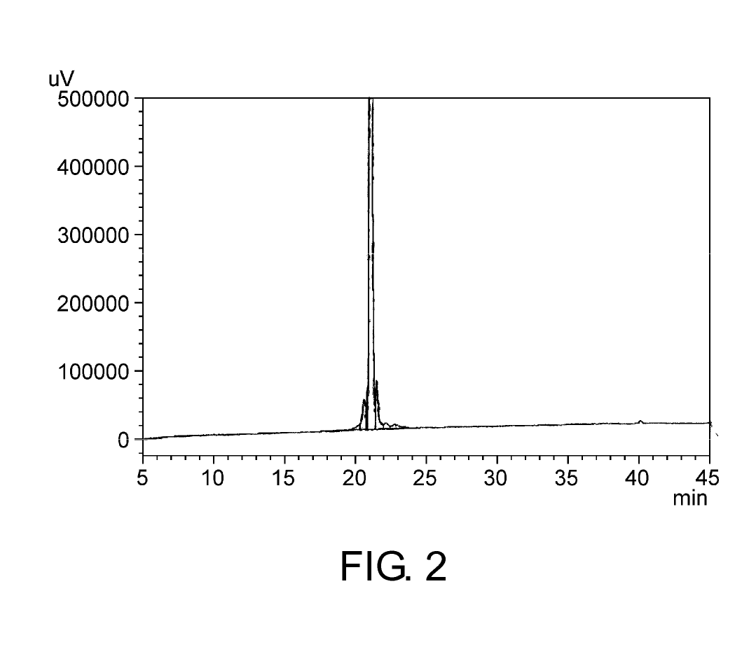

As an example, the chromatogram for degradation products obtained immediately after addition of sodium chloride (at the start) and that obtained after one week of preservation at 40° C. in the dark following addition of sodium chloride are shown in FIGS. 1 and 2, respectively.

The overall area of peaks excluding the main peak in each chromatogram was calculated based on the following formula.

[Area of peaks excluding the main peak]=[(overall area of all peaks)−(area of main peak)]/(overall area of all peaks)×100 Formula 1:

The shaded portion in FIG. 1 corresponds to the area of the main peak. This area represents the eMIP content. Therefore, the “area of peaks excluding the main peak” determined based on Formula 1 represents the amount of degradation product generated from eMIP.

The percentages of degradation product generated from eMIP immediately after addition of an additive (at the start) and after one week of preservation at 40° C. in the dark following addition of the additive are listed in Tables 4 and 5 for each of the 36 types of additives.

TABLE 4
Various additives and percentage of degradation product
generated from eMIP (1)

| | | | Degradation product (%) | | |
|---|---|---|---|---|---|
| | | | | After | Amount |
| No. | Category | Additive | At the start | preservation | increased |
| 0 | — | none | 10.2 | 21.4 | 11.2 |
| 1 | Salt | Sodium chloride | 10.1 | 13.3 | 3.2 |
| 2 | | Calcium chloride hydrate | — (*) | — (*) | — |
| 3 | | Magnesium chloride | — (*) | — (*) | — |
| 4 | Sugar | D-Mannitol | 10.2 | 18.0 | 7.8 |
| 5 | | D-Sorbitol | 10.1 | 19.0 | 8.9 |
| 6 | | Fructose | 10.1 | 86.4 | 76.3 |
| 7 | | Lactose hydrate | 10.2 | 29.1 | 18.9 |
| 8 | | Sucrose | 10.0 | 18.9 | 8.9 |
| 9 | | Glucose | 10.0 | 61.8 | 51.8 |
| 10 | | Glycerin | 10.1 | 35.7 | 25.6 |
| 11 | Amino acid | Glycine | 10.2 | 17.3 | 7.1 |
| 12 | | L-Alanine | 9.9 | 15.4 | 5.5 |
| 13 | | L-Histidine | 10.4 | 11.4 | 1.0 |
| 14 | | L-Arginine | 10.4 | 12.5 | 2.1 |
| 15 | | L-Arginine hydrochloride | 10.1 | 12.4 | 2.3 |
| 16 | | Sodium L-glutamate | 10.0 | 16.8 | 6.8 |
| 17 | | L-Aspartic acid | 10.1 | 16.8 | 6.7 |
| 18 | | L-Cysteine | 11.1 | — (**) | — |
| 19 | | L-Lysine hydrochloride | 10.1 | 13.6 | 3.5 |

(*) The test was discontinued because the additive was insoluble in phosphate buffer at pH 7.
(**) The experiment was discontinued due to precipitation.

TABLE 5
Various additives and percentage of degradation product generated from eMIP (2)

| | | | Degradation product (%) | | |
|---|---|---|---|---|---|
| | | | | After | Amount |
| No. | Category | Additive | At the start | preservation | increased |
| 20 | Surfactant | Sorbitan sesquioleate | — (*) | — (*) | — |
| 21 | | Polyoxyethylene | — (*) | — (*) | — |
| | | hydrogenated castor oil 60 | | | |
| 22 | | Polyoxyethylene sorbitan | — (*) | — (*) | — |
| | | monolaurate | | | |
| 23 | | Polysorbate 20 | 10.4 | 79.6 | 69.2 |
| 24 | | Polysorbate 80 | 10.3 | 72.7 | 62.4 |
| 25 | | Macrogol 400 | 10.3 | 58.8 | 48.5 |
| 26 | | Polyoxyethylene (160) | 10.2 | 76.1 | 65.9 |
| | | polyoxypropylene (30) | | | |
| | | glycol | | | |
| 27 | Anti-oxidant | Ascorbic acid | 80.4 | — (***) | — |
| 28 | | Sodium hydrogen sulfite | — (**) | — (**) | — |
| 29 | | Sodium sulfite | — (**) | — (**) | — |
| 30 | | α-Thioglycerin | 10.6 | — (***) | — |
| 31 | | Cysteine hydrochloride | — (**) | — (**) | — |
| 32 | | Dried sodium sulfite | — (**) | — (**) | — |
| 33 | | Citric acid hydrate | 10.4 | 12.3 | 1.9 |
| 34 | | Sodium thioglycolate | 10.8 | — (***) | — |
| 35 | | Sodium pyrosulfite | — (**) | — (**) | — |
| 36 | Chelating agent | Sodium edetate (EDTA) | 10.3 | 12.8 | 2.5 |

(*) The test was discontinued because the additive was insoluble in phosphate buffer at pH 7.
(**) The test was discontinued because the sample was not eluted from the column due to adsorption.
(***) The experiment was discontinued due to precipitation.

Furthermore, the percentages of degradation product generated from eMIP at the start and after one week of preservation at 40° C. in the dark were also determined without adding any additive. The result is shown as No. 0 in Table 4.

According to Tables 4 and 5, the increase in the percentage of degradation product generated from eMIP was 11.2% without any additive. The eMIP degradation was significantly suppressed by the following additives: sodium chloride (Wako Pure Chemical Industries, Japanese Pharmacopoeia Grade), L-histidine (Wako Pure Chemical Industries, Wako special grade), L-arginine (Wako Pure Chemical Industries, Wako special grade), L-arginine hydrochloride (Wako Pure Chemical Industries, Wako special grade), L-lysine hydrochloride (Wako Pure Chemical Industries, special grade), citric acid (Wako Pure Chemical Industries, Japanese Pharmacopoeia Grade), and sodium edetate (EDTA) (Wako Pure Chemical Industries, Japanese Pharmacopoeia Grade, Lot No. WKG6966).

The percentage of increase of eMIP degradation product was markedly high (62.4%) when 1% Polysorbate 80 (NOF Corporation, polysorbate 80 (HX)) was added.

Furthermore, eMIP degradation was also enhanced (51.8%) upon addition of glucose (Wako Pure Chemical Industries, Japanese Pharmacopoeia Grade). Thus even though glucose infusion solutions are commonly used as a base for drip infusions, this reveals that the combined use of eMIP and glucose is inadvisable.

The above result demonstrated that, in a solvent of pH 5 where the solubility of eMIP was poor, the addition of sodium chloride or citric acid markedly improved the solubility.

In a solvent of pH 7 where the solubility of eMIP was higher but degradation of eMIP occurred, the stability against degradation was revealed to be improved by adding L-histidine, L-arginine, L-arginine hydrochloride, L-lysine hydrochloride, and sodium edetate (EDTA), in addition to sodium chloride and citric acid.

Thus, the eMIP degradation was demonstrated to be suppressed by adding at least one or more additives selected from sodium chloride, L-histidine, L-arginine, L-arginine hydrochloride, L-lysine hydrochloride, citric acid, and sodium edetate (EDTA). Furthermore, phosphate buffer of pH 5 to 7 was found to be a preferable pH adjuster for the aqueous eMIP preparations of the present invention.

### Example 3

### Assessment of the Effect of Non-Ionic Surfactants in Aqueous Preparations Comprising eMIP

Additives that suppress the adsorption of eMIP onto plastic or glass surfaces were assessed by the method described in EXAMPLE 2.

The result showed that the adsorption onto plastic or glass surfaces can be inhibited by adding polysorbates, which are non-ionic surfactants; however, degradation products were generated from eMIP when the polysorbate concentration was high (Table 5).

Thus, the concentrations of polysorbates added were examined. The result demonstrated that the adsorption and degradation of eMIP were suppressed when the weight % concentrations of polysorbates were 0.005 to 0.1%.

The pH of the above-described buffer was examined in the presence of Polysorbate 80™ at a weight % concentration of 0.01%. The result showed that eMIP was not precipitated and the adsorption onto apparatus surface can be inhibited within the pH range of 5.0 to 7.4.

### INDUSTRIAL APPLICABILITY

The present invention provides stable injections and drip infusions comprising eMIP.

1. An aqueous preparation comprising eMIP as an active ingredient, wherein the preparation comprises one or more additives selected from L-histidine, L-arginine, L-arginine hydrochloride, L-lysine hydrochloride, sodium chloride, citric acid, and sodium edetate (EDTA), and wherein the pH of the preparation is adjusted to 5 to 7.

2. The aqueous eMIP preparation of claim 1, wherein the pH adjustment is achieved by using a phosphate buffer.

3. The aqueous eMIP preparation of claim 1 or 2, wherein the concentration of eMIP as an active ingredient is within the range of 0.01 to 6.5 mg/ml and the concentration of the additives is within the range of 5 to 15 mg/ml in the aqueous preparation.
